(19) United States
(12) Patent Application Publication
(10) Pub. No.: US 2009/0235938 A1
(43) Pub. Date: Sep. 24, 2009

(54) Protective upper leg sleeve

(76) Inventor: Monty B. Roach, Weimar, CA (US)

Correspondence Address: Walt Froloff, 273D Searidge Rd, Aptos, CA, 95003, US

(21) Appl. No.: 12/381,014

(22) Filed: Mar. 6, 2009

Related U.S. Application Data
(60) Provisional application No. 61/037,797, filed on Mar. 19, 2008.

(51) Int. Cl. A61F 13/00

(52) U.S. Cl. 128/891; 128/892

(57) ABSTRACT

The present invention describes a protective thigh sleeve for preventing and protecting skin from irritation and rash developing as a result of physical activities from skin surface friction.

### BACKGROUND

1. Field of the Invention

The present invention relates generally recreational gear, and particularly to a protective sleeve to aid in the prevention of skin irritation and skin rash from physical activities such surfing, skim boarding, wake boarding, body boarding, boogie boarding, body surfing, jet skiing, windsurfing, jogging and snorkeling scuba diving.

Many people engaged in recreational activities such as but not limited to surfing, skim boarding, wake boarding, body boarding, boogie boarding, body surfing, jet skiing, windsurfing and snorkeling scuba diving incur skin irritation that forms into a rash when working in jean pants and also during aquatic activities in board shorts or trunks. Often rash develops, between the thighs and also on the back of the thigh leg, the hamstring area.

Many of the big surf industry manufacturers produce attractive and quick drying board shorts. However, wearing these board shorts, during physical activities does not stop skin irritation and subsequent skin rash. For some, this rash can develop when snorkeling for even an hour or so. The kicking back and forth leg motion for a continuous amount of time with board shorts rubs the back of the legs raw and a rash is formed. This quickly impedes further enjoyment and generally ceases any further physical activity.

Occasionally, even jeans can rub the inner thigh raw and prevent walking normally. The pain of rubbing raw inner thigh is immense and usually accompanied by a red rash.

A current remedy is to rub Vaseline on the high friction surface area. This is a very untidy solution compounded by the embarrassment of application generally in a open recreational environment. Another current solution is spandex shorts. These are very uncomfortable in warm areas because they amplify the heat of friction on the thighs. During aquatic activities most will refuse to wear spandex shorts under board shorts. When water is temperature is better than warm, wearing two pairs of shorts quickly becomes untenable. This is compounded as spandex shorts have a tendency to hike up on the leg and expose the lower half of the hamstring. This allows the rash to take on the lower part of the hamstring were the an aquatic board short will rub against the leg. When this happens, the one not enjoying the recreating activity is frustrated and quits.

What are needed are way to reduce friction between rubbing skin surfaces without retaining more heat in the friction heated zone.

### SUMMARY

The present invention discloses a protective thigh sleeve for preventing and protecting skin from irritation and rash developing as a result of physical activities from skin surface friction. The protective sleeve comprises a thin stretchable water compatible material tapered sleeve, with all sleeve seam lines smooth and flush with the inside sleeve surface and sleeve fitted dimensions to snuggly cover the thigh girth, length extending from the upper thigh to the knee top. Whereby one wearing sleeves will experience drastically reduced friction on protected skin surfaces. The protective thigh sleeve provides protection from wearing outer garments. The sleeve material is made substantially of polyester, spandex, lycra or combinations.

### BRIEF DESCRIPTION OF DRAWINGS

Specific embodiments of the invention will be described in detail with reference to the following figures.

FIG. 1 illustrates an upper leg view of the protective sleeve in accordance with an embodiment of the invention

FIG. 2 shows a wearer installing the protective sleeve in accordance with an embodiment of the invention

FIG. 3 illustrates a swimmer using the protective sleeve in accordance with an embodiment of the invention

### DETAILED DESCRIPTION

In the following detailed description of embodiments of the invention, numerous specific details are set forth in order to provide a more thorough understanding of the invention. However, it will be apparent to one of ordinary skill in the art that the invention may be practiced without these specific details. In other instances, well-known features have not been described in detail to avoid unnecessarily complicating the description.

Objects and Advantages

There are several objects or advantages of the present invention.

It is therefore an object of the invention to help prevent rash development during physical activities, including aquatic, recreational and sporting activities. Examples of activities that will benefit from the instant invention are but not limited to surfing, skim boarding, wake boarding, body boarding, boogie boarding, body surfing, jet skiing, windsurfing and snorkeling scuba diving.

It is another object of the invention to assist in uncomfortable hot days where work pants and or denim jean pants can rub inner thighs raw. Embodiments of the invention will prevent inner thigh from becoming sensitive to a rash. Giving the wearer the protection from the sweat and garments rubbing against the inner thighs or other skin surfaces.

It is another object of the invention to manufacture size embodiments conforming to individuals of leg sizes, shapes and geometries in need of friction and rash prevention during physical activity causing skin friction and rash.

It is yet another object of the invention to produce a variety of embodiments in various colors and printed patterns, for more stylish and fad applications.

### EMBODIMENTS OF THE INVENTION

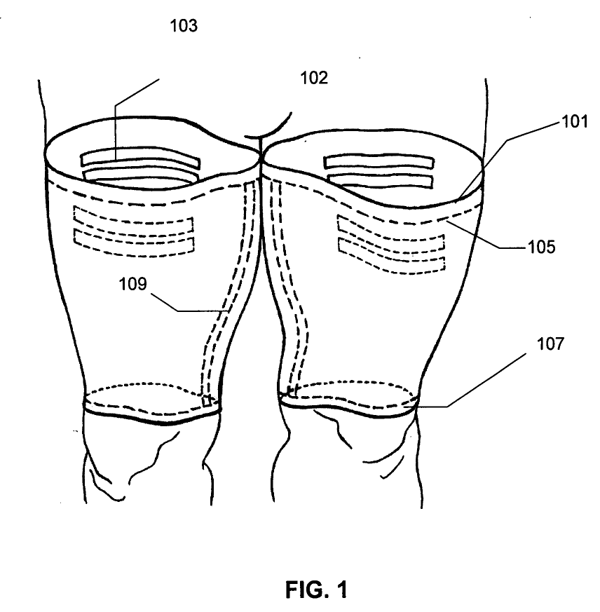

FIG. 1 illustrates a perspective view of a protective sleeves in accordance with an embodiment of the invention. This protected sleeve embodiment is tapered to fit the upper leg region of a typical limb, 8 to 10 inches in length, tapered to fit from a large diameter edge 101 to a small diameter edge 107. Unless anchored, sleeves will slowly work their way down with repetitive movement by some physical activities. Thus, an aspect of the invention supplies the anchor to the skin by way of stay riser 103, two are shown on each sleeve. These strips 103, speed bumps for sleeve movement, are of positioned laterally to the leg axis or the most likely travel direction of the sleeve during leg motion. The strip 103 material increases the frictional force to resist slippage down the leg. This material can be a flowable silicone elastomer which is screen printed on the inside of the sleeve. Screen printing is a good way to apply the flowable silicone elastomer of sleeve stretchable water compatible material. The flowable silicone elastomer stretches with the main sleeve material. The main sleeve material is stretchable generally man made material like polyester, spandex, lycra, etc. Without the sleeve strip anchor, activity tends to work the sleeve into a bunch on the thigh and decreases effectiveness. The top 101 as well as the bottom edges 107 have ⅝ inch hem seams 105. The lateral seam 109 can be ¼ inch flat felt for good results.

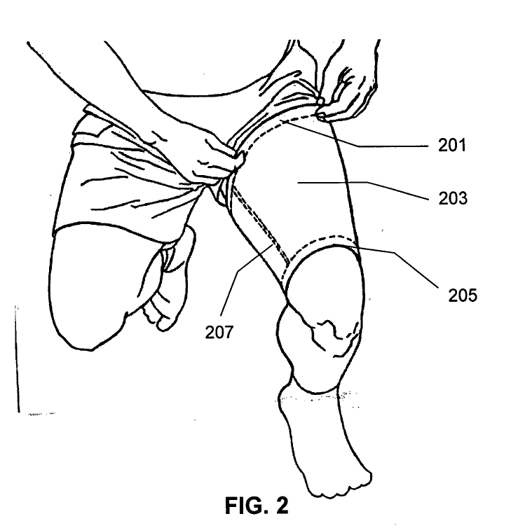

FIG. 2 illustrates a perspective right leg view of the protective sleeve in accordance with an embodiment of the invention. FIG. 2 shows the sleeves use under clothing, as even shorts will cause skin irritation with activity. A sleeve embodiment material 203 is stretchable and comfortable to the skin, covering from the buttocks line 201 to the top of the knee 205. This gives the wearer a thin skin layer between the pants or board shorts being worn that can produce skin irritation. Taper shaping the sleeve garment can be done by coupling a flat curved trapezoidal piece equal length sides to a Flat Felt seam 207. This will ensure against protruding edge or excess garment material to cause skin irritation. The Hem Seam on top and the bottom is a standard ⅝″ Hem Seam 16. The overall length 18 of the finished sleeve is 10″ in length. This length will cover most if not all of the upper leg and aide in the event of skin irritation or rash development.

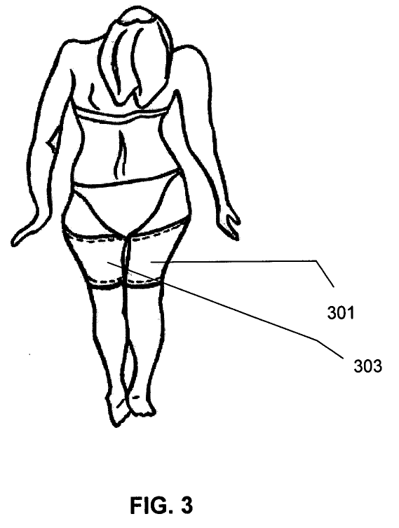

FIG. 3 illustrates a perspective view of the protective sleeve installation and position on the leg in accordance with an embodiment of the invention. The swimmer shown from the back top view is swimming with the protective sleeve 301 302 on each upper leg. The embodiment shown can be worn plainly as shown or under sports clothing, not shown.

Therefore, while the invention has been described with respect to a limited number of embodiments, those skilled in the art, having benefit of this invention, will appreciate that other embodiments can be devised which do not depart from the scope of the invention as disclosed herein. Other aspects of the invention will be apparent from the following description and the appended claims.

What is claimed is:

1. A protective upper leg sleeve for preventing skin friction irritation and skin rash from developing as a result of physical activity, comprising:
a thin stretchable water compatible material tapered sleeve;
at least one riser strip coupled to the inside of the sleeve lateral to the sleeve tapered axis;
sleeve seam lines made smooth and flush with the inside sleeve surface and edges, and
sleeve fitted dimensions to snuggly cover the thigh girth, sleeve length extending from the upper thigh to the knee top,
whereby one wearing sleeves will experience drastically reduced friction on protected skin surfaces.

2. The protective thigh sleeve for preventing skin friction irritation and skin rash as in claim 1 further comprising protection from wearing outer garments.

3. The protective thigh sleeve for preventing skin friction irritation and skin rash as in claim 1 wherein the sleeve material is made substantially of polyester, spandex, lycra, other artificial material or combinations.

4. The protective thigh sleeve for preventing skin friction irritation and skin rash as in claim 1 further comprising a flowable silicone elastomeric material band coupled transverse to the leg and to the inside of the sleeve surface.

5. The protective thigh sleeve for preventing skin friction irritation and skin rash as in claim 1 wherein sleeve edges are ⅝″ hem for smooth finish hem seam.

6. The protective thigh sleeve for preventing skin friction irritation and skin rash as in claim 1 wherein ¼″ hem for smooth finish hem seam is conformably compatible to a axial Flat Felt Seam.
